(19) United States
(12) Patent Application Publication
(10) Pub. No.: US 2020/0033345 A1
(43) Pub. Date: Jan. 30, 2020

(54) METHOD AND KIT FOR DIAGNOSIS AND/OR PROGNOSIS OF NON-HEMATOLOGICAL TUMORS

(71) Applicants: Natalia MALARA, Lamezia Terme (IT); Vincenzo MOLLACE, Bianco (IT)

(72) Inventor: Natalia MALARA, Lamezia Terme (IT)

(21) Appl. No.: 16/491,187

(22) PCT Filed: Mar. 6, 2018
(86) PCT No.: PCT/IB2018/051438

§ 371 Date: Sep. 5, 2019

(30) Foreign Application Priority Data
Mar. 6, 2017 (IT) 102017000024609

(51) Int. Cl. G01N 33/574; C12N 5/09; G01N 33/50

(52) U.S. Cl. CPC G01N 33/57407; C12N 5/0693; G01N 33/5091; C12N 2500/34; C12N 2501/91; C12N 2500/84; C12N 2501/11; C12N 2501/115; C12N 2500/38

(57) ABSTRACT

The present invention relates to an in vitro method for the diagnosis and/or prognosis of non-haematological tumours by analysing circulating tumour cells isolated from a blood sample or a derivative thereof and to a kit for such purpose. The present invention further relates to a specific culture medium particularly effective for use in such method.

The present invention relates to an in vitro method for the diagnosis and/or prognosis of non-haematological tumours by analysing circulating tumour cells isolated from a blood sample or a derivative thereof and to a kit for this purpose. The present invention further relates to a specific culture medium, particularly effective for use in such method.

### DESCRIPTION OF PRIOR ART

Notwithstanding the several screening programmes there is still a high cancer mortality rate. The current methods for cancer detection vary depending upon the involved organ. The current methods for example include biopsy, faecal occult blood test, colonoscopy, computed tomography, magnetic resonance (“MRI”), X-ray mammography and ultrasounds. The current diagnostic techniques often are capable of detecting tumours only in advanced state, moreover they are often invasive and expensive methods.

The circulating tumour cells (“CTC”) are at very low levels, it is estimated that there is one in a billion of blood cells, and most of them die during circulation. However, a significant portion of these rare cells survives, then there is a considerable interest in the use thereof for the diagnosis and disease prognosis and response to treatment. However, the detection of this population of rare cells is technically still very challenging.

For example, as far as specifically the patients affected by intracranial cancers are concerned, the presence of the hematoencephalic barrier is known which mediates and surveys the bidirectional traffic from systemic circulation to brain tissue. Most part of brain tumours develops in childhood, they are solid and appear anatomically and histologically different (Stiller C A et al 1994). One of the most complex ones among this group is the family of gliomas. These tumours can arise in any area of the central nervous system; they have various histological features which can differ histologically even inside the tumour mass itself; and they can metastasize if benign or malignant. Gliomas which are localized in the encephalic trunk, in turn, constitute a specific nosological entity. 15-20% of these tumours are astrocytomas clinically characterized by particular semeiotics, they are glial tumours having low degree of growth and generally they have an indolent and slow course (Walker D A et al 2004). The remaining 80% of these tumours are diffused and involve the bridge. Tissue biopsy cannot be performed for the critical localization of these tumour entities which jeopardize noble brain areas with not precisely computable sequences. Currently in most paediatric patients in which the histological diagnosis cannot be performed, diagnosis is based upon information found with RNM (magnetic nuclear resonance). Recently, the solid tumours positioned in different portions of human body have been studied for an intrinsic peculiarity thereof, that of releasing tumour cells in the peripheral blood.

Several experimental studies are shown in literature about the procedures for isolating and selecting the Circulating Tumour Cells (CTC). Commonly adopted procedures, for example, provide the use of sorter and/or machinery such as conventional cytofluorimetry in which isolation takes place by means of specific antibodies which recognize markers selected on CTC (Terstappen et al 1998), or with vital cytofluorimetry, however up to know implemented exclusively in laboratory animal and which consists in the injection of fluorescent ligands capable of recognizing the tumour cells which are subsequently quantified during their passage in the surface blood vessels (He et al 2007). Such technique in humans is still under development even if the use of specific ligands has already produced encouraging results (He et al 2009). CTC quantification can be performed also by using immunomagnetic spheres (Zieglschmid et al 2005) or with sophisticated imaging systems by means of digital microscopic apparatuses (Hisieh et al 2006, Kraeft et al 2004 e Krivacic et al 2004). In particular, for CTCs automatic digital microscopy systems have been described (Mesker et al 2006).

The presence of epithelial tumour cells has been demonstrated in the peripheral blood of patients subjected to high-dose chemotherapy (Weaver et al 1998, Ross et al 1993) through immunochemical assays and by means of cell culture tests (Pedrazzoli et al 2000). In particular, the procedure described by Pedrazzoli suggests the possibility of using cell cultures to detect CTCs.

The greatest problem associated to isolation and then, to the subsequent quantization and/or characterization of the epithelial tumour cells, is their low number. In particular, it is estimated that in a patient with metastases originated from a solid tumour there is one CTC every 10⁹ cells existing in a blood sample. In 2010 the working group of Lu (Lu et al 2010) showed a procedure for enriching CTC, isolated from patients with breast cancer, providing their in vitro cultivation for a maximum period of time of 12 hours.

The standard technique used in most laboratories consists in CTC isolation after separating the blood sample on suitable gradient. In particular, for the gradient usually a branched synthetic copolymer, having high molecular weight and being very water soluble (d=1.077 g/mL), is selected, synthetized starting from sucrose and epichlorohydrin the trade name thereof is Ficoll®. Such procedure provides the phase recovery with a gradient value between 1,057 and 1,069 Kg/m³ which is considered to be the phase in which there are the mononuclear cells, thus circulating tumour cells (CTC) included.

The recovery of the above particular phase for recovering CTCs is strongly rooted in the practice of the person skilled in the art, as indicated and shown in any laboratory manual (Lu et al 2009, Paterlini-Brechot et al 2007, mention the reference in full).

From the known state of art in primis a problem emerges from all of them and that is on one side the fact of obtaining a sufficient number of CTCs from a blood sample and on the other side the fact of isolating a fraction reflecting the real heterogeneity of such cell population.

Such problem was mainly faced by trying to enrich the isolated cell sample by means of culture passages. In particular, such method exploits the greater proliferative capability characterizing the tumour cells with respect to the normal haematological cells, thus allowing the exponential increase in their number in cell culture to the detriment of the normal cells. However, as it is a primary cell line, an enrichment thereof is linked to the fact that the cell growth/duplication can be obtained only within few days due to the short half-life characterizing the in vitro primary cultures themselves.

Even if more sophisticated techniques (sorter/cytofluorimetry) have been developed with the purpose of obtaining a sufficient number of CTCs, however they have several disadvantages such as the high cost of machinery, overall dimension of the same, use by specialized technical personnel, thus in other words they are techniques which are not likely used in most laboratories with base instrumentation. Moreover, these methods use monoclonal antibodies or a pool di monoclonal antibodies directed against a particular antigen or group of antigens expressed by CTCs, it follows that the CTCs which do not express such antigens, but which however are present in circulation, are excluded by the selection and, thus, they are not present in the isolated sample which then does not result to represent the heterogeneity of the real population of tumour cells circulating in a subject.

The object of the present invention is to propose a new and original solution to the problems highlighted above existing in the state of known art.

### BRIEF DESCRIPTION OF THE FIGURES

FIGS. 1 and 2 show table 1 and table 2 wherein the estimate of the circulating tumour cells is shown, obtained with the method traditionally used for isolating CTC cells from blood and the number of CTC cells isolated with the herein described method, respectively.

FIG. 3: (a), (b), (c), (d), (e), (f) represent primary cultures of CTC derived from the blood of patients affected by colon (a), breast (b) and lung (c) cancers, whereas (d), (e), (f) primary cultures derived in equal experimental conditions from the blood of healthy subjects.

FIG. 4 shows the expression of calpain-2 in lung tumour cells (PT), lung circulating tumour cells (CTC) isolated according to the herein disclosed method, before (C1) and after (C2) the sample surgical removal.

FIG. 5. Analysis of the pre-cultivation phenotype. The graph relates to the cells collected after the passage in centrifuge and isolated from the phase.

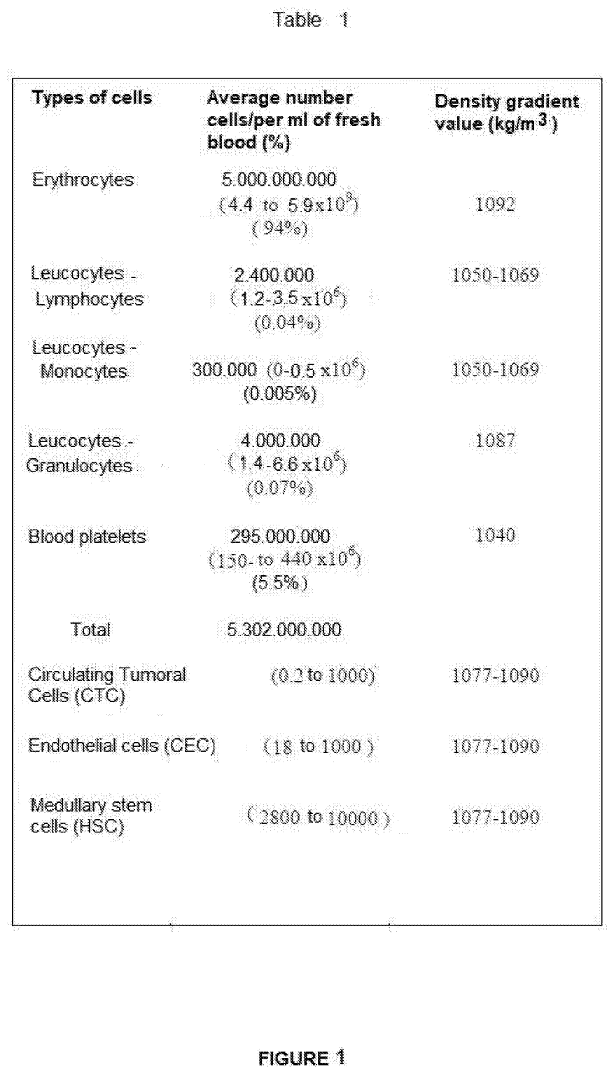

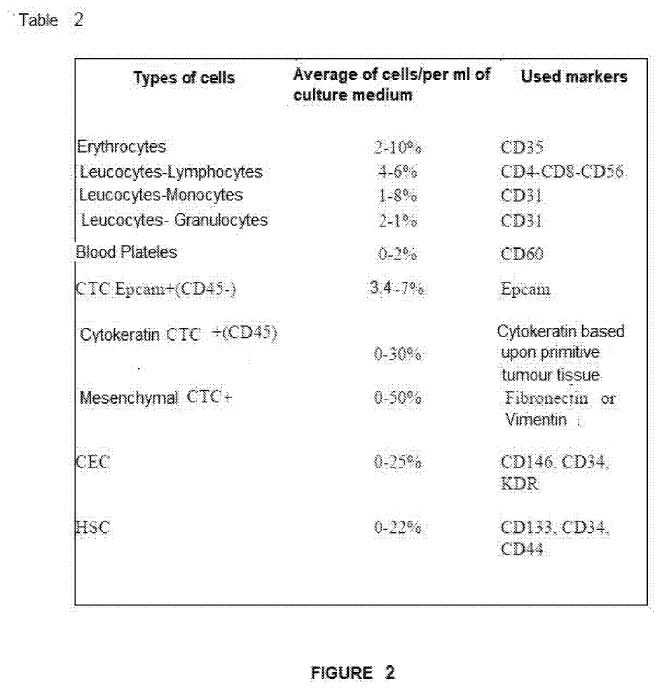

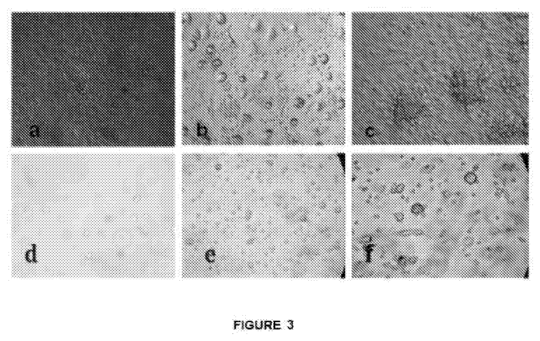

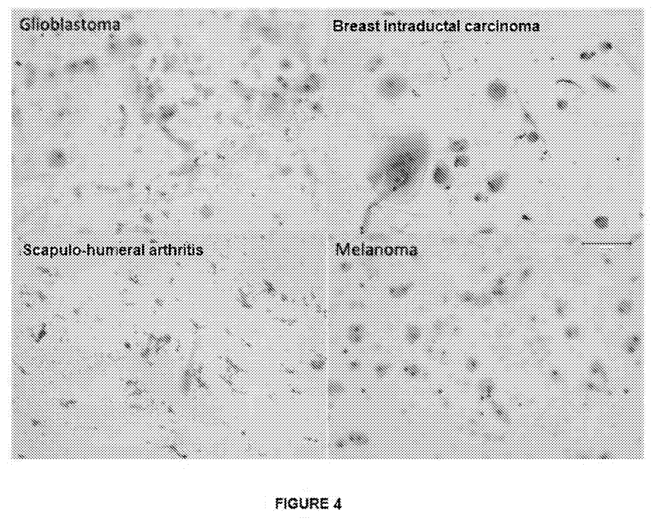

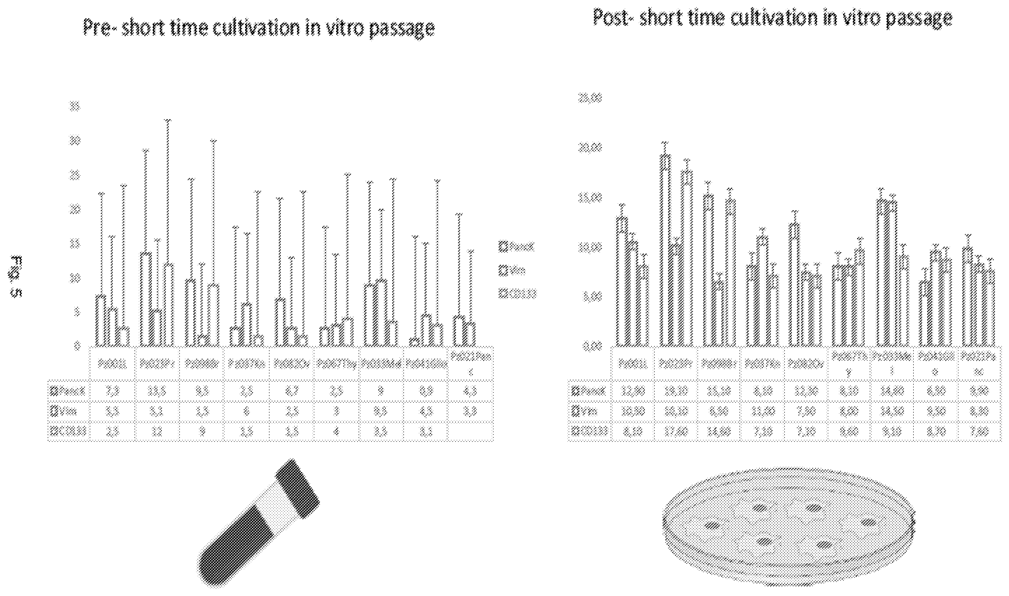

### SUMMARY OF THE INVENTION

For the first time an ameliorating method is herein described in terms of sensitivity and reproducibility for detecting the circulating tumour cells (CTC) in patients affected by cancer. The sensitivity of the illustrated method facilitates experiment repeatability. The routine isolation and the cultivation of malignant epithelial cells according to what herein described provides a better control of diagnosis and prognosis of tumour disease. Moreover, the herein described method provides information about feasibility of specific chemotherapy on the patient and based upon a simple in vitro test it allows to detect the heterogeneity of sensibility to drug. Moreover, the used culture medium has been optimized to increase the CTC yield in vitro both in adhesion and suspension. At last it was observed that the number of spontaneous formations of cell spheres increases as the disease stage progresses and therefore they are considered as a negative prognosis sign.

In the publication Malara et al. Journal of Biological Regulators & Homeostatic agents, 1 Jan. 2014 717-731 the Protocol for culturing ctc from Non-Small Cell Lung Cancer (NCSLC) is described. With respect to the described method in this publication the use of optimized culture mediums allows to obtain circulating tumour cells for the diagnosis and/or prognosis in patients with non-haematological tumours of different type not only with NCSLC. The culture medium composition was tested in order to avoid qualitative discrepancies between the subset types of CTC found after culture and those observed in the same sample before in vitro passage. The medium type and the time of 14 days modify the absolute number of CTCs per single phenotypic subset not modifying the phenotypic inter subset proportions. In other words, the number of cells is modified but they are not modified qualitatively.

Firstly, the present invention then relates to an in vitro method for the diagnosis and/or prognosis of a not-haematological tumour by means of analysing the circulating tumour cells isolated from a blood sample or a derivative thereof as defined by claim 1.

Secondly, the present invention relates to a cell culture medium as defined by claim 9.

Thirdly, the present invention relates to a kit for the diagnosis and/or prognosis of a not-haematological tumour by means of analysing circulating tumour cells isolated from a blood sample or a derivative thereof as defined by claim 10.

Preferred features of the present description are set forth in the relative depending claims.

### DETAILED DESCRIPTION OF THE INVENTION

An in vitro method for the diagnosis and/or prognosis of non-haematological tumours by analysing circulating tumour cells is herein disclosed. The method provides a first isolation passage of circulating tumour cells from a blood sample, or a derivative thereof, which will be performed according to what described in the patent application ITRM20120028 shown herein too for sake of completeness.

Under blood derivative in the present description a derivative is meant obtained from blood, for example, by filtration and/or natural separation such as in case of plasma, pleural or ascites fluid.

The separation of a blood sample with Ficoll is conventionally performed by a person skilled in the art in his/her daily work and described in any laboratory manual, therefore it does not require additional examination herein. Preferably, such separation is performed by centrifugation. In an embodiment of the present invention, the first passage of the method for isolating CTCs is obtained by centrifugation for about 20 minutes at 4° C.

As a consequence of the just described procedure the different components of the sample of interest separate along the gradient according to their specific density value. The inventor of the present method has surprisingly detected a phase of Ficoll gradient therealong preferentially CTCs concentrate. Such phase of Ficoll gradient corresponds to the phase with a density value comprised between about 1,080 and 1,090 (kg/m³), phase which has never been described or suggested in literature as useful to isolate circulating tumour cells. In particular, the state of prior art designates as value useful to the above purpose a value ranging between 1,050 and 1,070 (kg/m³).

Then, the second passage of the herein described method consists in collecting from said separated sample on Ficoll, as previously indicated, the phase with density value comprised between 1,080 and 1,090 (kg/m³). Optionally, the so-collected phase can be diluted with a suitable solution, wherein under suitable a solution is meant which does not induce any type of chemical/physical alteration to the portion of up-to-now separated and collected sample. Examples of such solutions are phosphate-buffered saline or a solution of standard citrate of saline solution (CSD), solution of stringent washing (pH 7.0 at 71° C., 0.06 M of NaCl, 6 mm C₆H₅Na₃O₇.2H₂O) Subsequently, the collected gradient phase is subjected to additional separation, preferably by centrifugation. In an embodiment of the present invention, such centrifugation is performed at about 1,500 rmp preferably for 10 minutes and at room temperature.

At last, the so-obtained pellet is recovered. Such pellet corresponds to the circulating tumour cells, thus to the CTCs of a patient and circulating.

In particular, the isolating method as described herein characterizes for a yield relatively to the number of isolated CT which is of about 10⁵ tumour circulating cells/ml of blood or a derivative thereof.

The method provides an additional passage in which the isolated circulating tumour cells as described above are cultured in a medium optimized for such purpose comprising:

Nutrient mixture supplemented with Foetal bovine serum

Heparin, in particular Heparin 25000;

Epidermal growth factor;

Fibroblast growth factor;

Bovine serum albumin;

D-Glucose,

L-ascorbic acid;

one or more antibiotics selected from penicillin, streptomycin and/or amphotericin.

According to an embodiment the medium will include D-glucose in a concentration comprised between 4 and 10 mM, in particular 5.55 mM and ascorbic acid in a concentration comprised between 5 and 20 mM, in particular 14 mM.

According to an embodiment the nutrient mixture supplemented with Foetal bovine serum is of Ham F-12 type. Hereinafter the composition of a commercial medium of F-12 Ham, commercialized by Sigma-Aldrich, is shown:

| | N4888 | N6658 | N6760 |
|---|---|---|---|
| Component | [1x] g/L | [1x] g/L | g/L |
| Inorganic Salts | | | |
| Calcium Chloride | 0.0333 | 0.0333 | 0.0333 |
| Cupric Sulfate•5H₂O | 0.0000025 | 0.0000025 | 0.0000025 |
| Ferrous Sulfate•7H₂O | 0.000834 | 0.000834 | 0.000834 |
| Magnesium Chloride | 0.0576 | 0.0576 | 0.0576 |
| Potassium Chloride | 0.224 | 0.224 | 0.224 |
| Sodium Bicarbonate | 1.176 | 1.176 | — |
| Sodium Chloride | 7.599 | 7.599 | 7.599 |
| Sodium Phosphate Dibasic | 0.14204 | 0.14204 | 0.14204 |
| (anhydrous) | | | |
| Zinc Sulfate•7H₂O | 0.000863 | 0.000863 | 0.000863 |
| Amino Acids | | | |
| L-Alanine | 0.009 | 0.009 | 0.009 |
| L-Arginine•HCl | 0.211 | 0.211 | 0.211 |
| L-Asparagine•H₂O | 0.01501 | 0.01501 | 0.01501 |
| L-Aspartic Acid | 0.0133 | 0.0133 | 0.0133 |
| L-Cysteine•HCl•H₂O | 0.035 | 0.035 | 0.035 |
| L-Glulamic Acid | 0.0147 | 0.0147 | 0.0147 |
| L-Glulamine | — | 0.146 | 0.146 |
| Glycine | 0.00751 | 0.00751 | 0.00751 |
| L-Histidine•3HCl•H₂O | 0.02096 | 0.02096 | 0.02096 |
| L-Isoleucine | 0.00394 | 0.00394 | 0.00394 |
| L-Leucine | 0.0131 | 0.0131 | 0.0131 |
| L-Lysine•HCl | 0.0365 | 0.0365 | 0.0365 |
| L-Methionine | 0.00448 | 0.00448 | 0.00448 |
| L-Phenylalanine | 0.00496 | 0.00496 | 0.00496 |
| L-Proline | 0.0345 | 0.0345 | 0.345 |
| L-Serine | 0.0105 | 0.0105 | 0.0105 |
| L-Threonine | 0.0119 | 0.0119 | 0.0119 |
| L-Tryptophan | 0.00204 | 0.00204 | 0.00204 |
| L-Tyrosine•2Na•2H₂O | 0.00778 | 0.00778 | 0.00778 |
| L-Valine | 0.0117 | 0.0117 | 0.0117 |
| Vitamins | | | |
| D-Biotin | 0.0000073 | 0.0000073 | 0.000073 |
| Choline Chloride | 0.01396 | 0.01396 | 0.01396 |
| Folic Acid | 0.00132 | 0.00132 | 0.00132 |
| myo-Inositol | 0.018 | 0.018 | 0.018 |
| Niacinamide | 0.000037 | 0.000037 | 0.000037 |
| D-Pantothenic Acid (hemicalcium) | 0.00048 | 0.00048 | 0.00048 |
| Pyridoxine•HCl | 0.000062 | 0.000062 | 0.000062 |
| Riboflavin | 0.000038 | 0.000038 | 0.000038 |
| Thiamine•HCl | 0.00034 | 0.00034 | 0.00034 |
| Vitamin B₁₂ | 0.00136 | 0.00136 | 0.00136 |
| Other | | | |
| D-Glucose | 1.802 | 1.802 | 1.802 |
| Hypoxanthine | 0.00408 | 0.00408 | 0.00408 |
| Linoleic Acid | 0.000084 | 0.000084 | 0.000084 |
| Phenol Red•Na | 0.0013 | 0.0013 | 0.0013 |
| Putrescine•HCl | 0.000161 | 0.000161 | 0.000161 |
| Pyruvic Acid•Na | 0.11 | 0.11 | 0.11 |
| Thioctic Acid | 0.00021 | 0.00021 | 0.00021 |
| Thymidine | 0.00073 | 0.00073 | 0.00073 |
| Add | | | |
| L-Glutamine | 0.146 | — | — |
| Sodium Bicarbonate | — | — | 1.176 |

According to an additional embodiment the medium will include or will consists of:

Nutrient mixture supplemented with Foetal bovine serum, in particular Ham F-12 (commercially available for example by Sigma Aldrich catalogue number N3520) supplemented with Foetal bovine serum at 10%:

Heparin 25000 u/5 ml (fc: 0.5 U/ml);

epidermal growth factor (EGF) 200 μg/ml (fc: 50 ng/ml);

fibroblast growth factor (FGF) 25 μg/ml (fc: 25 ng/ml)

bovine serum albumin (BSA) 1%,

D-Glucose 5.55 mM,

L-ascorbic acid 14 mm;

Solution of penicillin-streptomycin 1% and/or amphotericin B 0.1%.

The method provides an additional passage of cytological analysis of the cells cultivated with the purpose of performing a diagnosis and/or prognosis of tumour of the patient therefrom the blood sample was collected. The cytological analysis preferably will be a microscope analysis and/or an analysis by means of cytofluorimetry, for example by means of Fluorescence-activated cell sorting (FACS).

The method could be used to obtain circulating tumour cells deriving from a tumour of solid type or from epithelial cells. For example stromal cancers, cardiac myxomas, intracranial cancers including the multiform glioblastoma, thyroid cancers, adrenal cancers, pancreatic, colon, breast, stomach cancers, cholangiocarcinomas, melanoma, spinocellular and basal cancers and lung small cell cancers. The present description further relates to a kit for the diagnosis and/or prognosis of non-haematological tumours by analysing circulating tumour cells comprising the culture medium as described herein. The kit could further comprise at least one tube wherein the phase with a density value comprised between about 1.080 and 1.090 (kg/m³) is highlighted, and at least a solution for carrying out the Ficoll gradient. Preferably such tube is a sterile tube having capacity suitable to contain a quantity of blood sample or a derivative thereof sufficient to perform the separation according to the above method. The tube characterizes in having the area corresponding to the phase of Ficoll gradient comprised between about 1,080 and 1,090 (kg/m³) limited visibly so as to allow and lead the operator towards a correct isolation of the tumour circulating cells according to the herein claimed method. The absence of highlighting the phase 1,080 and 1,090 (kg/m³) on the tube would imply the collection of phases characterized by the presence of blood components, such as for example, lymphocytes and monocytes, with consequent contamination of the obtained CTC sample. It appears clear that the quantity of the solution aliquot for carrying out the Ficoll gradient will be selected by taking into account the capacity and the number of tube included in the above kit.

The kit can further comprise at least an operating means useful to perform the isolation of the circulating tumour cells according to the herein disclosed method. Therefore, such means can be selected in the group comprising a: tube, pipette, tube with EDTA, syringe, needle, phosphate buffered saline and sterile water.

### Experimental Section and Examples

### Procedure

### Selection of Patients

### Cancer Patients

1. Caucasic people
2. Age within 18 and 65 years old
3. Cancer diagnosis

### Exclusion Criteria:

1. The patients with infective active states
2. The subjects not without food
3. The individuals subjected to pharmacological treatment for at least 48 hours Collection of blood samples
1) The blood (5 ml) collected from each donor in tubes for the blood collection including EDTA. In order to avoid contamination of blood samples with epithelial skin cells, collect the second tube once the first blood sample has been collected.
CRITICAL PASSAGE: Overturn gently the blood tube 10 times and keep it at room temperature. The processes within maximum 4 hours after blood collection.
All subsequent passages are at room temperature (20-22°) in sterile hood at room temperature
2) mix gently 5 ml of blood with 3 ml of dilution buffer (PBS1×) ATTENTION: for point 4) use 10-ml pipettes (Corning cat.n. cc4488) for reducing cell lysis.

### CELL SEPARATION BY CENTRIFUGATION IN DENSITY GRADIENT TIME: MORE THAN 50 MIN

5) apply carefully 4 ml of cell suspension on 3 ml of Ficoll density gradient. The cell suspension should float above the gradient.
6) centrifuge the gradient at 1,840 rpm in a centrifuge swinging bucket for 25 min at 22°. Note: transfer even the tube with PCM medium kept at 4° at room temperature ready for passage 10.
7) Collect the wished fraction with a pipette. In particular, of fraction 2, the most opaque band includes fragments of cells, the circulating not-haematological cells, the haematological cells. The fraction 1 includes debris, the fraction 3 includes mainly monocytes and lymphocytes, the fraction 4 or pellet mainly includes red blood cells. Collect the fraction 3 for the maximum yield of cells.
8) dilute the gradient, mix the cell suspension with a ratio of 1:2 with washing buffer (PBS 1×).
9) Centrifuge the cell suspension including the wished fraction for 10 min. at 20°, 1860 rpm per minute in a centrifuge swinging bucket. Repeat twice.
10) Eliminate the supernatant including debris. Remove the pellet of cells by moving the tube bottom with a finger. Immediately suspend again the cells in 1 ml of PCM
11) Count the cells under an inverted microscope by using 200-ul of cell suspension staining with trypan blue.
CRITICAL PHASE: cover and put the tube in CO₂ incubator to avoid toxic exhalations of pH in CO₂ environment

### Plate Culture

2) Plate and cultivate cells.
Follow option A for the cells adhering to the culture system and option B for the spheres (A) In order to favour the adherence to the cell culture plate the cells at wished concentration in COMPLETE PCM are placed in wells or in 60 mm×15 mm culture plate. Preferably 24-well plates will be used to ease the management in several points of time or concentrations and the widespread of contaminations is less probable.
13) continue the culture for the subsequent 5-7 days, provide new nutriment and remove waste by removing half of medium on day 4 or 5 and replace it with an equal volume of complete medium. Repeat this medium change every 3 days.
14) after 4-5 days the adherent cells can be collected. Different types of cells can be identified by specific immunostainings or through evaluation by cytometry. Such culture also includes endothelial cells and lymphocytes.

### Option (B) for Sphere Cancer

After 4-5 days the spheres can be collected, disaggregated (by using a 1-ml pipette) for the characterization in cytometry or for sorter of epithelial tumour cells. The selected CTC can be planted again for proliferation or differentiation of additional tumour spheres. The density is 1000 cells/ml.

### Composition of the Optimized Culture Medium

Hereinafter the composition of the culture medium is illustrated which resulted to be the most effective one:

Nutrient mixture Ham F-12 (available on the market for example by Sigma Aldrich catalogue number N3520) supplemented with Foetal bovine serum at 10%:

Heparin 25000 u/5 ml (fc: 0.5 U/ml);

epidermal growth factor (EGF) 200 μg/ml (fc: 50 ng/ml);

fibroblast growth factor (FGF) 25 μg/ml (fc: 25 ng/ml)

bovine serum albumin (BSA) 1%,

D-Glucose 5.55 mM,

L-ascorbic acid 14 mm;

Solution of penicillin-streptomycin 1% and/or amphotericin B 0.1%.

1. An in vitro method for the diagnosis and/or prognosis of non-hematological tumors comprising the following steps:
i) Isolating a population of circulating tumor cells from a blood sample, or a derivative thereof, obtained from a patient suffering from, or potentially suffering from tumor by means of the following steps:
a) separating said blood sample or derivative thereof on a Ficoll gradient by centrifugation;
b) collecting from said separated sample the phase with a density value comprised between 1,080 and 1,090 (kg/m3) and diluting the collected phase
c) centrifuging the collected and diluted phase; and
d) recovering the obtained pellet
ii) cultivating the pellet obtained in step d) in a culture medium containing:
Nutrient mixture supplemented with fetal bovine serum
Heparin;
Epidermal growth factor;
Fibroblast growth factor;
Bovine serum albumin;
D-Glucose,
L-ascorbic acid; and
one or more antibiotics selected from penicillin, streptomycin and/or amphotericin; and
iii) performing a cytological analysis of cells cultured in step ii) so as to provide a diagnosis and/or prognosis of said cancer.

2. The method according to claim 1, wherein said circulating tumor cells derive from a solid tumor or are epithelial cells.

3. The method according to claim 1, wherein said non-hematological tumor is selected from the group consisting of: stromal cancers, cardiac myxomas, intracranial cancers including multiform glioblastoma, thyroid cancers, adrenal cancers, pancreatic cancers, colon cancers, breast cancers, stomach cancers, cholangiocarcinomas, melanoma, spinocellular cancers and basal cancers and lung small cell cancers.

4. The method according to claim 1, wherein the sample of said blood derivative is a sample of whole blood, pleural or ascites fluid.

5. The method according to claim 1, wherein said centrifugation at said step a) is carried out at 700 g for 20 minutes at 4° C. or at 1,840 rpm in a centrifuge swinging bucket for 25 minutes at 22° C.

6. The method according to claim 1, wherein said centrifugation at step d) is carried out at 1850 or 1860 rpm for 10 minutes at room temperature.

7. The method according to claim 1, wherein the D-glucose in said medium is in a concentration between 4 and 10 mM and/or ascorbic acid is in a concentration between 5 and 20 mM.

8. The method according to claim 1, wherein said medium comprises:
Nutrient mixture Ham F-12 supplemented with fetal bovine serum at 10%:
Heparin 25000 u/5 ml (fc: 0.5 U/ml);
epidermal growth factor (EGF) 200 μg/ml (fc: 50 ng/ml);
fibroblast growth factor (FGF) 25 μg/ml (fc: 25 ng/ml);
bovine serum albumin (BSA) 1%,
D-Glucose 5.55 mM,
L-ascorbic acid 14 mm; and
penicillin and/or streptomycin and/or amphotericin B.

9. A medium for culturing circulating tumor cells from a blood sample, or a derivative thereof, comprising
Nutrient mixture supplemented with fetal bovine serum
Heparin;
Epidermal growth factor;
Fibroblast growth factor;
Bovine serum albumin;
D-Glucose,
L-ascorbic acid; and
one or more antibiotics selected from penicillin, streptomycin and/or amphotericin.

10. The medium for culturing circulating tumor cells from a blood sample according to claim 9 wherein the D-glucose in said medium is in a concentration between 4 and 10 mM.

11. The medium for culturing circulating tumor cells from a blood sample according to claim 9 wherein ascorbic acid is in a concentration between 5 and 20 mM.

12. The medium for culturing circulating tumor cells from a blood sample according to claim 9, wherein said medium comprises the following mixture:
Nutrient mixture Ham F-12 supplemented with fetal bovine serum at 10%;
Heparin 25000 u/5 ml (fc: 0.5 U/ml);
epidermal growth factor (EGF) 200 μg/ml (fc: 50 ng/ml);
fibroblast growth factor (FGF) 25 μg/ml (fc: 25 ng/ml);
bovine serum albumin (BSA) 1%,
D-Glucose 5.55 mM,
L-ascorbic acid 14 mm; and
penicillin and/or streptomycin and/or amphotericin B.

13. A kit for the diagnosis and/or prognosis of non-hematological tumors comprising the culture medium according to claim 9 and at least one aliquot of a solution for carrying out the Ficoll gradient and/or at least one tube wherein the phase with a gradient value between 1,080 and 1,090 (kg/m3) is highlighted and/or at least one means selected from the group consisting of: a tube, pipette, tube with EDTA, syringe, needle, aliquot with phosphate buffered saline and sterile water aliquot.

14. The method according to claim 7, wherein the D-glucose concentration in said medium is 5.55 mM and/or the ascorbic acid concentration is 14 mM.

15. The medium according to claim 10, wherein the D-glucose in said medium is at a concentration of 5.55 mM.

16. The medium according to claim 11, wherein the ascorbic acid is at a concentration of 14 mM.
